(19) United States
(12) Patent Application Publication
(10) Pub. No.: US 2005/0037069 A1
(43) Pub. Date: Feb. 17, 2005

(54) Solid and stable creatine/citric acid composition(s) and compositions carbohydrate(s) or hydrates thereof, method for the production and use thereof

(76) Inventors: Martin Purpura, Bonn (DE); Ivo Pischel, Rossbach (DE); Ralf Jager, Freising (DE); Gunter Ortenburger, Trostberg (DE)

Correspondence Address: FULBRIGHT & JAWORSKI, LLP, 666 FIFTH AVE, NEW YORK, NY, 10103-3198, US

(21) Appl. No.: 10/495,827

(22) PCT Filed: Nov. 29, 2002
(86) PCT No.: PCT/EP02/13518

(51) Int. Cl. A61K 31/715; A61K 9/20

(52) U.S. Cl. 424/464; 514/54

(57) ABSTRACT

A description is given of formulations comprising solid and stable creatine/citric acid composition(s) and carbohydrate(s) or hydrates thereof having a weight ratio of the constituents creatine/citric acid composition(s) and carbohydrate(s) of 1:0.01 to 1.0. Carbohydrate components, which can be used in anhydrous form, as hydrates or as moist products, are in principle all polyhydroxyaldehydes and polyhydroxyketones. The creatine component is preferably used in anhydrous form or in aqueous alcoholic or organic solution. The formulations should preferably be prepared by reacting the carbohydrates in the form of solutions, suspensions or as solid substance with the creatine/citric acid composition or solution thereof at −20 to 120° C. and, if appropriate, finally removing the liquid phase, or else by a thermal treatment, a treatment with ultrasound or microwave of mixtures consisting of the creatine component and solid carbohydrates, preferably in a vacuum. This produces formulations comprising creatine/citric acid compositions and carbohydrate which can be used, inter alia, to increase endurance and strength in the sports sector, for weight reduction and body fat reduction, in the health sector, for health disorders in women, and also as a food supplement and for animal nutrition.

The present invention relates to a formulation which comprises one or more solid and stable creatine/citric acid compounds and one or more carbohydrates or hydrates thereof, a process for production thereof and use thereof.

Creatine salts are known in the most varied forms and contain the physiologically harmless creatine cation of the formula (I)

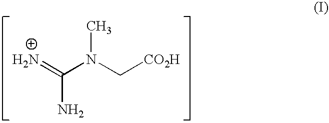

Creatine is not only an endogenous substance and a valuable food supplement, but also possesses valuable therapeutic properties. Creatine has been known for over 100 years as a muscle substance, with it acting as energy source for the muscles. In a series of scientific studies, it was found that taking creatine can lead to an increase in muscle mass and muscle performance. There are also scientific findings that, under the influence of creatine, the pancreas increases release of insulin. Insulin promotes the uptake of glucose and amino acids into the muscle cells and stimulates protein synthesis. Furthermore, insulin decreases the protein breakdown rate.

Citric acid is a tricarboxylic acid which is known to occur in the plant kingdom and also in many animal tissues and body fluids. Citric acid is included, inter alia, among the number of prominent compounds which participate in the physiological oxidation of fats, proteins and carbohydrates to form carbon dioxide and water (see, inter alia, tricarboxylic acid cycle).

Citric acid, which was first isolated in 1784 from lemon juice by the Swedish chemist Karl Wilhelm Scheele is currently prepared biotechnologically by fermenting cane sugar or molasses by the mold Aspergillus niger. Citric acid is used in its natural form or as food additive E 330 in the formulation of foods, but also in baking powders, effervescent lemonades and generally for acidification and as a taste compound. In addition, citric acid increases the stability of foods or other organic substances.

Pure anhydrous citric acid forms mostly colorless rhombic crystals having a slightly acidic taste.

U.S. Pat. No. 5,973,199 discloses, inter alia, isolated water-soluble salts of creatine and citric acid which have a melting point of 112 to 114° C. This creatine citrate is prepared by suspending citric acid monohydrate in ethyl acetate, then adding creatine monohydrate and then mixing the mixture for 4 hours at 25° C. This produces a product which contains approximately 90% creatine citrate having a melting point of between 112 and 114° C. and varying amounts of solvent.

Likewise previously described are di- and tricreatine citrates (U.S. Pat. No. 6,211,407), the melting points of which are said to be 146° C. and 154° C., respectively, and which have a creatine content of 57.7 and 67.2% by weight, respectively. Details of the structure of such salts, however, cannot be inferred from this document.

Effervescent formulations which comprise creatine citrate as 1:1 salt or physical mixtures of creatine and citric acid are disclosed by U.S. Pat. No. 5,925,378.

Many creatine salts, such as, inter alia, the creatine pyruvates, creatine ascorbates, and also creatine citrates, have valuable physiological, therapeutic and dietetic properties. Most of the known creatine salts are used, for example, to increase endurance and strength in the sports sector, for reducing weight and body fat and also as protective substance for body cells and body tissue, in particular the cardiovascular, hepatic, nephrotic, peritoneal and neuronal tissue, as a substance for inhibiting free-radical formation, and also as free-radical scavenger in body cells and body tissues (including synovial tissue), and furthermore in the health sector and as food supplements.

Creatine pyruvates, which, as pure substance, are compounds stable at temperatures below 40° C., are typical for the physical properties of many creatine salts. However, if creatine pyruvates are exposed to temperatures above 40° C., or if formulations containing creatine pyruvates are prepared with addition of certain nonstabilizing substances, a rapid breakdown of the creatine pyruvates may be observed.

Thus the storage stability of the creatine pyruvates, like also other salts of creatine with in particular organic acids, especially at relatively high temperatures, is usually not very pronounced, which is believed to be also due to the fact that, for example, the pyruvates are unstable 2-oxocarboxylic acid salts and, furthermore, most of the known salts of creatine readily break down with formation of creatinine. Since creatine, in addition, is present as an internal salt, and is only a weak base, the storage stability, especially over a relatively long time period, is adversely affected. In particular, combinations of creatine compounds, for example the citrates, with compounds having a tendency to instability, lead to quality losses for these reasons.

WO 98/28263 discloses pyruvates in the form of storage-stable creatine compounds, which can be combined in physiologically compatible compositions with, in addition to pharmaceutical formulation aids, inter alia, also carbohydrates, such as glucose, dextrose and maltose.

Combinations of, inter alia, creatine pyruvates with carbohydrates, such as sucrose, fructose, glucose, and aspartame, which act as sweeteners, are disclosed by U.S. Pat. No. 5,886,040.

Creatine-containing formulations consisting of a neuro-transmitter, α-lipoic acid and optionally L-carnitine, coenzyme Q and other physiologically active additives, for example sugars, are described by DE-A 198 30 768, to which these formulations can comprise in particular creatine pyruvate and, inter alia, are also said to be storage stable.

Finally, DE-A 198 36 450 discloses stable formulations comprising pyruvic acid (salts) for animal nutrition which, in addition to three other component groups, also comprise physiologically valuable additives, such as carbohydrates. Further components proposed are organic acids, for example citric acid, and aminocarboxylic acids, for example creatine. Preference as pyruvic acid salt is given to creatine pyruvate.

Although the four last-mentioned documents disclose combinations of creatine salts with carbohydrates, and other formulations are described which can comprise inter alia creatine, citric acid and also carbohydrates, there is no indication that the storage stability and other properties can be improved by compositions consisting of creatine and citric acid.

The object was therefore to develop composition(s) in a readily meterable form and in which the disadvantages of the prior art are at least in part eliminated.

This object was achieved by a solid and stable formulation comprising creatine/citric acid composition (s) and carbohydrate(s) or their hydrates having a weight ratio of the constituents creatine/citric acid composition(s) and carbohydrate(s) of 1:0.01 to 1.0.

It has now surprisingly been found that creatine/citric acid composition(s), when they are formulated with carbohydrates, are extremely temperature-stable and storage-stable, even when other compounds which are unstable and/or are destabilizing are added. This increased storage stability even at elevated temperatures of, for example, ≧30° C. and, in particular, ≧35° C., even for a long period of time, is displayed by a reduced formation of creatinine as degradation product. Preferably, the inventive formulations have, after storage at 40° C. for 28 days, a creatinine content of ≦500 ppm, particular preferably ≦350 ppm.

The inventive formulation thus makes a considerably longer storage life possible of the compositions in question which consist of creatine and citric acid, and also an increased ability to withstand stress, with respect to temperature and duration, not only during processing, but also during storage, which makes accessible, inter alia, for example novel preparation and/or formulation processes.

Since these formulations comprising creatine/citric acid composition(s), on account of their stability, have a considerably reduced proportion of unwanted degradation product and byproduct and physiologically unsuitable constituents, they can be very readily metered and have a high compatibility.

In practice, formulations which have proved to be very suitable are those whose constituents creatine/citric acid composition(s) and carbohydrate(s) are present in a weight ratio of 1:0.01 to 0.5 and, very particularly preferably, in a weight ratio of about 1:0.1. Preferred embodiments of the claimed formulations have a water content up to 30% by weight.

Particularly suitable creatine components which have been proved in practice are those which comprise, as creatine/citric acid composition(s), physiologically compatible salts, additions and complexes in a molar ratio of creatine to citric acid of 1.0 to 3.0:1.0. Those which also come into question are, inter alia, creatine/citric acid compounds which react in a labile manner to external influences. However, their combinations with stable creatine/citric acid compositions are comprised by the invention.

To stabilize the formulations, in principle, any carbohydrates can be used, for example all polyhydroxyaldehydes (aldoses) and polyhydroxyketones (ketoses) and also their derivatives, as a base. According to the present invention, however, particularly the aldohexoses and ketohexoses and also the oligosaccharides or polysaccharides derived therefrom and O-glycosidic derivatives thereof have proven themselves. In particular, monosaccharides, for example glucose, fructose, ribose and sorbitol and/or cyclic and open-chain oligohexoses having from 2 to 10 monomer units, such as sucrose, lactose and maltose, but also polysaccharides having 11 to 106 monomer units, for example inulin, arabinogalactan, maltodextrins, starch, chitosan and glycogen, are preferred. It is important that any desired mixtures of carbohydrates can also be used.

The present invention, however, also takes into account formulations which, in addition to the components essential to the invention, in addition comprise other creatine-containing compositions, in particular creatine salts, such as creatine pyruvates and creatine ascorbates. However, proteins, for example collagen or whey powder, and suitable acids, for example ascorbic acid, can also reinforce the stabilizing action of the carbohydrate components.

Formulations which have proved to be particularly suitable are those which are prepared by the following processes: according to a first embodiment, the carbohydrate(s) are reacted in the form of solutions, suspensions or as solid substance with the creatine/citric acid composition in the temperature range from −20 to 120° C., and preferably between 10 and 90° C., whereupon the resultant solution is optionally finally freed from the liquid phase, which is preferably performed by solid/liquid separation, distillation, evaporation and/or concentration, in each case in a vacuum, spray-drying or freeze-drying or by any other suitable separation method. According to a second embodiment, a mixture consisting of the acidic creatine/citric acid component or its solution and the solid carbohydrate component is subjected in the temperature range from −20 to 120° C. to a thermal treatment, a treatment with ultrasound and/or microwave, which can also be performed in a vacuum, and leads to the formation of homogeneous formulations.

All these reactions can be carried out in the presence of a solvent or diluent or else can be carried out free from solvent. Solvents or diluents which have proved to be suitable for preparing the claimed aggregates are all customary solvents such as water, alcohols, ethers, ketones, esters, organic acids, aliphatic and aromatic hydrocarbons.

Of course, the desired formulations can also be obtained by simple dry mixing of the components.

According to the invention, the carbohydrate component can be used in anhydrous form, as hydrate or as moist product for forming the formulation. The acidic creatine salt component can likewise be used in anhydrous form, in aqueous solution or dissolved in one of the abovementioned alcoholic or organic solvents or diluents. All claimed reaction variants can be performed in the known processing apparatuses, such as in kneaders, mixers, paddle driers, stirred vessels and also spray driers.

The inventive formulations comprising creatine/citric acid composition(s) and carbohydrate are obtained in this manner in high yield and purity without further necessary purification steps.

The present invention thus provides that either both starting components are used in solid or liquid form, or that one component is solid and the other liquid. It is only necessary to pay attention to the fact that, for the inventive formulations, the creatine/citric acid component and the carbohydrate component are always used in the context of the claimed weight ratios.

On account of their excellent properties, the inventive formulations are used for pharmaceutical purposes or in the food industry, for example for increasing endurance and strength in the sports sector, for weight reduction and body fat reduction, and also as a protective substance for body cells and body tissue, particularly cardiovascular, hepatic, ophthalmic, nephrotic and neuronal and also transplant tissue, and as a substance for the inhibition of free-radical formation and also as free-radical scavenger substance in body cells and body tissues (also synovial tissue), in the health sector, in particular in the treatment of obesity and overweight, in skin disorders, for wound healing and for lowering blood pressure, and also in health disorders in women which are caused especially by changes in the female sex hormone level, for example manifestations of premenstrual syndrome (PMS) dysmenorrhea, climacteric, in meteorosensitivity, migraines, tension headache, sleeplessness and affective disorders and as food supplement additive. The use of the aggregates in animal nutrition is also provided according to the invention.

The inventive formulations can be administered in the form of tablets, powders, dragees, capsules, drinks and gels, and in particular as effervescent tablets and effervescent powders, with this series being able to be extended as desired.

The present invention is thus an important advance in the use of creatine/citric acid compositions in the therapeutic and dietetic sectors and also in sports medicine. This is because, on account of the prior art, it was unforeseeable that solid and storage-stable formulations comprising creatine/citric acid composition (s) and carbohydrate are accessible by simple methods and lead to readily meterable products. Furthermore it has been found that the creatine/citric acid compositions undergo no, or at most only very slight, condensation and decomposition reactions during preparation of the formulations. Furthermore, the claimed formulations comprising creatine/citric acid composition(s) and carbohydrate are successfully prepared without subsequent necessary workup. Aqueous solutions containing the creatine component, or solvents, can also be used, formulations of defined water of crystallization content then being obtained.

It remains to be stated that the present invention, in particular using the preparation variants which are likewise claimed, does not comprise mere mixtures of the components essential to the invention creatine/citric acid composition(s) and carbohydrate(s), as are already sufficiently known from the prior art, but the claimed formulations are actual physical or chemical reaction products.

The examples hereinafter illustrate the advantages of the present invention.

### EXAMPLES

A creatine-citric acid compound (3:1) (KC) as pure substance and mixtures of this compound (3:1) with 1, 5 and 10% by weight addition of a carbohydrate (CH) were stored for 28 days at 60° C. and then the contents of the creatine decomposition product creatinine were determined. The comparison is against the initial data of the reference substance KC which is stable under cooling or up to room temperature (RT) and having a creatine content of 67.1% by weight and a creatinine content of <67 ppm (limit of detection).

| | Creatinine | |
|---|---|---|
| | content | Observa- |
| | [ppm] | tions |
| Addition: 1% | | |
| KC without CH; 40° C., 28 days | 770 | white |
| KC with glucose; 40° C., 28 days | 300 | white |
| KC with sucrose; 40° C., 28 days | 300 | white |
| KC with arabinogalactan; 40° C., 28 days | 292 | white |
| KC with sucrose and whey powder; 40° C., | 270 | white |
| KC with sucrose and collagen; 40° C., | 309 | white |
| 28 days | | |
| KC with lactose; 40° C., 28 days | 330 | white |
| KC with maltodextrin; 40° C., 28 days | 300 | white |
| KC with glucose and ascorbic acid; 40° C., | 300 | white |
| 28 days | | |
| KC with starch; 40° C., 28 days | 273 | white |
| Addition: 5% | | |
| KC without CH; 40° C., 28 days | 770 | white |
| KC with glucose; 40° C., 28 days | 300 | white |
| KC with sucrose; 40° C., 28 days | 264 | white |
| KC with arabinogalactan; 40° C., 28 days | 302 | white |
| KC with sucrose and whey powder; 40° C., | 242 | white |
| 28 days | | |
| KC with sucrose and collagen; 40° C., | 311 | white |
| 28 days | | |
| KC with lactose; 40° C., 28 days | 301 | white |
| KC with maltodextrin 40° C., 28 days | 300 | white |
| KC with glucose and ascorbic acid; 40° C., | 300 | white |
| 28 days | | |
| KC with starch; 40° C., 28 days | 257 | white |
| Addition: 10% | | |
| KC without CH; 40° C., 28 days | 770 | white |
| KC with glucose; 40° C., 28 days | 300 | white |
| KC with sucrose; 40° C., 28 days | 225 | white |
| KC with arabinogalactan 40° C., 28 days | 266 | white |
| KC with sucrose and whey powder; 40° C., | 303 | white |
| 28 days | | |
| KC with sucrose and collagen; 40° C., | 352 | white |
| 28 days | | |
| KC with lactose; 40° C., 28 days | 298 | white |
| KC with maltodextrin 40° C., 28 days | 300 | white |
| KC with glucose and ascorbic acid; 40° C., | 300 | white |
| 28 days | | |
| KC with starch; 40° C., 28 days | 262 | white |

1. A formulation comprising a solid and stable creatine/citric acid composition and a carbohydrate component or hydrate thereof, characterized in that the weight ratio of the constituents creatine/citric acid composition and carbohydrate is from 1:0.01 to 1.0 and in that the formulation after storage at 40° C. for 28 days has a creatinine content of ≦500 ppm.

2. The formulation as claimed in claim 1, wherein the creatine/citric acid composition and the carbohydrate component are present in a weight ratio of 1:0.01 to 0.5.

3. The formulation as claimed in claim 1 comprising up to 30% by weight of water.

4. The formulation as claimed in claim 1, wherein said creatine/citric acid is selected from physiologically compatible salts, additions and complexes in a molar ratio of creatine to citric acid of 1.0 to 3.0:1.0.

5. The formulation of claim 1, wherein said carbohydrate component is at least one of the members selected from the group consisting of polyhydroxyaldehyde and polyhydroxyketones.

6. The formulation as claimed in claim 1, wherein said carbohydrate component is an aldohexose, a ketohexose nor an oligosaccharide, polysaccharide or O-glycosidic derivate thereof.

7. The formulation as claimed in claim 1 wherein said carbohydrate component is an open-chain oligohexose having 11 to 10⁶ monomer units.

8. The formulation as claimed in claim 1 further comprising other creatine compositions, particularly preferably creatine salts, such as creatine pyruvates and creatine ascorbates, and/or a protein component and/or an acid component, such as collagen or ascorbic acid.

9. A process for preparing a formulation comprising a solid and stable creatine/citric acid composition and a carbohydrate component or hydrates, thereof having a weight ratio of creatine/citric acid composition and carbohydrate of 1:0.01 to 1.0 comprising reacting the carbohydrate component, in the form of a solution or suspension or as solid substance, with the creatine/citric acid composition in the temperature range from −20 to 120° C., and removing the liquid phase.

10. The process as claimed in claim 9, wherein the liquid phase is removed by solid/liquid separation, distillation, evaporation and/or concentration in each case in vacuum, spray drying or freeze drying.

11. A process for preparing a formulation as claimed in claim 1 by thermal treatment, treatment with ultrasound or microwave of mixtures consisting of the creatine/citric acid composition or its solution and the solid carbohydrate component in the temperature range from −20 to 120° C.

12. The process as claimed in claim 11, wherein the treatment is performed under vacuum.

13. The process as claimed in claim 8, wherein the carbohydrate component is in anhydrous form, a hydrate or is a moist product.

14. The process as claimed in claim 8, wherein the creatine/citric acid composition is used in anhydrous form or in aqueous, alcoholic or organic solution.

15. The process as claimed in claim 8, wherein the solvent is at least one member selected from the group consisting of water, alcohols, ethers, ketones, esters, organic acids and aliphatic or aromatic hydrocarbons and also mixtures thereof.

16. The process as claimed in claim 9, wherein the preparation is carried out in kneaders, mixers, paddle driers, stirred vessels or spray driers.

17. A pharmaceutical food composition comprising the formulation of claim 1 and a pharmaceutical adjuvant or food product.

18. A method of increasing endurance and strength in the sports sector, for weight reduction and body fat reduction, and also as a protective substance for body cells and body tissue, particularly cardiovascular, hepatic, ophthalmic, nephrotic and neuronal and also transplant tissue, and as a substance for the inhibition of free-radical formation and also as free-radical scavenger substance in body cells and body tissues (also synovial tissue), in the health sector, in particular in the treatment of obesity and overweight, in skin disorders, for wound healing and for lowering blood pressure, and also in health disorders in women, for example manifestations of premenstrual syndrome (PMS) dysmenorrhea, climacteric, in meteorosensitivity, migraines, tension headache, sleeplessness and affective disorders, as food supplement and also in animal nutrition comprising administering the formulation of claim 1 to a subject in need thereof in an amount sufficient to achieve a desired result.

19. The composition of claim 18 in the form of tablets, powders, dragees, capsules, drinks and gels, effervescent tablets, and effervescent powders.
